(19) United States
(12) Patent Application Publication
(10) Pub. No.: US 2026/0364255 A1
(43) Pub. Date: Nov. 19, 2026

(54) LARGE VOLUME DRUG DELIVERY DEVICE

(71) Applicant: WEST PHARMA. SERVICES IL, LTD., Ra'anana (IL)

(72) Inventors: Gil YIGAL, Gav Yavne (IL); Menachem PERETZ, Kfar Saba (IL); Igor ISAKOV, Rehovot (IL); Edan SHALEV, Kfar Saba (IL); Yossi BAR EL, Beit Arie (IL)

(21) Appl. No.: 19/121,054

(22) PCT Filed: Oct. 26, 2023
(86) PCT No.: PCT/IB2023/060830

§ 371 Date: Apr. 14, 2025

Related U.S. Application Data
(60) Provisional application No. 63/381,029, filed on Oct. 26, 2022.

(51) Int. Cl. A61M 5/172; A61M 5/142

(52) U.S. Cl. CPC A61M 5/172; A61M 5/142

(57) ABSTRACT

Disclosed are devices and methods related to delivering medicament to a patient. A drug delivery device can include a control assembly and a plug-in assembly. The control assembly has a first connector and a motor to rotate the first connector. The plug-in assembly has a transfer needle to receive a medicament therein, a chamber to receive the medicament from the transfer needle, a piston movable within the chamber, and a second connector operably connected to the piston. The plug-in assembly is removably couplable to the control assembly, such that when the plug-in assembly is coupled to the control assembly, the first connector is operably coupled to the second connector. When the plug-in assembly is coupled with the control assembly, and when the motor rotates the first connector, the second connector also rotates, and the piston is moved axially within the chamber.

### CROSS REFERENCE TO RELATED APPLICATIONS

This application claims the benefit of U.S. Provisional Patent App. No. 63/381,029, filed Oct. 26, 2022, the disclosure of which is hereby incorporated by reference herein.

### TECHNICAL FIELD

This disclosure generally relates to drug delivery devices and related methods, and more particularly relates to a new design for administering large volumes of drug to patients.

### BACKGROUND

Medications can be delivered via pumps to patients at different volumes and rates. Many existing drug pumps are designed to deliver small volumes of medicament. Attempting to use existing pumps to deliver greater volumes results in more frequent pump cycles, which raises the temperature of the device due to friction caused during the pumping steps. The increase in pump temperature can damage sensitive medications, such as protein drugs that can be denatured. Pump components are often in contact with the medicament, thus jeopardizing sterility. These components also cannot be reused in future injections. Therefore, there is a need for an improved pump for delivering large volumes of drug with a reusable pump component that does not contact the medicament and that does not generate excess heat.

### SUMMARY OF THE INVENTION

The foregoing needs are met by the various aspects of the disclosed drug delivery devices. According to one aspect, a drug delivery device for administering a medicament to a patient includes a control assembly and a plug-in assembly. The control assembly has a first connector and a motor configured to rotate the first connector. The plug-in assembly has a transfer needle configured to receive a medicament therein, a chamber configured to receive the medicament from the transfer needle, a piston movable within the chamber, and a second connector operably connected to the piston. The plug-in assembly is removably couplable to the control assembly, such that when the plug-in assembly is coupled to the control assembly, the first connector is operably coupled to the second connector. When the plug-in assembly is coupled with the control assembly, and when the motor rotates the first connector, the second connector also rotates, and the piston is moved axially within the chamber.

According to another aspect of the disclosure, a drug delivery system includes a control assembly, a plug-in assembly, a medicament container, and an infusion patient interface. The control assembly has a first connector and a motor configured to rotate the first connector. The plug-in assembly has a transfer needle configured to receive a medicament therein, a chamber configured to receive the medicament from the transfer needle, a piston movable within the chamber, and a second connector operably connected to the piston. The medicament container has the medicament therein and is couplable with the plug-in assembly such that the transfer needle is in fluid communication with the medicament in the medicament container. The infusion patient interface is configured to contact an injection site and to deliver the medicament to the injection site from the chamber. The plug-in assembly is removably couplable to the control assembly, such that when the plug-in assembly is coupled to the control assembly, the first connector is operably coupled to the second connector. When the plug-in assembly is coupled with the control assembly, and when the motor rotates the first connector, the second connector also rotates, and the piston is moved axially within the chamber.

According to another aspect of the disclosure, a method of injecting a medicament using a drug delivery device is described. The drug delivery device has a plug-in assembly removably couplable to a control assembly. The method includes the steps of introducing a medicament container into the drug delivery device; coupling the plug-in assembly to the control assembly such that a first connector on the control assembly is operably engaged with a second connector on the plug-in assembly, and such that a transfer needle on the plug-in assembly is in fluid communication with the medicament in the medicament container; and actuating a motor to cause rotation of the first connector in a first rotational direction.

According to another aspect of the disclosure, a method of assembling a drug delivery device is described. The method includes introducing a medicament container into the drug delivery device; coupling the plug-in assembly to the control assembly such that a first connector on the control assembly is operably engaged with a second connector on the plug-in assembly, and such that a transfer needle on the plug-in assembly is in fluid communication with the medicament in the medicament container; disposing a patient interface onto an injection site; and coupling the patient interface with the plug-in assembly.

### BRIEF DESCRIPTION OF DRAWINGS

The present application is further understood when read in conjunction with the appended drawings. For the purpose of illustrating the subject matter, exemplary aspects of the subject matter are shown in the drawings; however, the presently disclosed subject matter is not limited to the specific methods, devices, and systems disclosed. In the drawings:

FIG. 1 depicts a schematic of a drug delivery device according to an aspect of this disclosure;

FIG. 2 depicts an angled view of a drug delivery device according to another aspect of this disclosure;

FIG. 3 depicts a cross-sectional view of a control assembly according to an aspect of this disclosure;

FIG. 4 depicts another cross-sectional view of the control assembly of FIG. 3;

FIG. 5 depicts another cross-sectional view of the control assembly of FIG. 3;

FIG. 6 depicts an angled view of a plug-in assembly according to an aspect of this disclosure;

FIG. 7 depicts another angled view of the plug-in assembly of FIG. 6;

FIG. 8 depicts a cross-sectional view of the plug-in assembly of FIG. 6;

FIG. 9 depicts a cross-sectional view of a plug-in assembly with the piston in a first position according to an aspect of this disclosure;

FIG. 10 depicts a cross-sectional view of a plug-in assembly with the piston in a second position according to an aspect of this disclosure;

FIG. 11 depicts a process of using a drug delivery device according to an aspect of this disclosure;

FIG. 12 depicts an angled view of a drug delivery device according to another aspect of this disclosure where the plug-in assembly is engaged with the control assembly;

FIG. 13 depicts an angled view of the drug delivery device of FIG. 12 with the plug-in assembly disengaged from the control assembly; and

FIG. 14 depicts a cross-sectional view of the plug-in assembly of the drug delivery device of FIG. 12.

Aspects of the disclosure will now be described in detail with reference to the drawings, wherein like reference numbers refer to like elements throughout, unless specified otherwise

### DETAILED DESCRIPTION OF ILLUSTRATIVE EMBODIMENTS

One or more medicaments can be delivered to a patient through a drug delivery device. The medicaments can be stored within the drug delivery device or be stored in a component operably connected to the drug delivery device. The drug delivery device typically includes a mechanism for delivering the medicament, which can include a pump, a motor, and the like, and typically includes a patient interface through which the medicament is delivered, which can include one or more needles, catheters, and the like. In some aspects, it may be desirable to reuse one or more components of the drug delivery device, for example, the electronic components. In such scenarios, the drug delivery device can include a disposable portion and a reusable portion. The disposable and reusable portions can be configured to be selectively engaged or disengaged from one another. Prior to use, the disposable and reusable portions can be engaged with one another to deliver the medicament to the patient; after use, the disposable portion can be separated from the reusable portion and disposed. The reusable portion can then be used again with another disposable portion.

In some circumstances, there may be a need to deliver a larger volume of medicament to a patient that exceeds the volumes of existing drug delivery devices. Embodiments described throughout this application disclose drug delivery devices configured to deliver larger volumes of medicament while retaining a relatively small size. The disclosed embodiments depict the drug delivery device as an assembly of at least two components, where one component may be intended to be reusable, and the other component may be intended to be disposable.

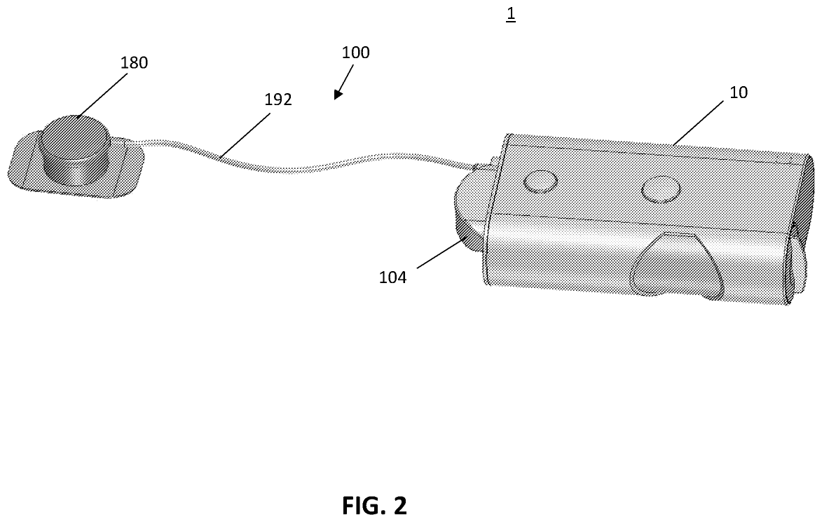

An exemplary embodiment of a drug delivery device 1 is depicted in FIG. 2. The drug delivery device can generally include a control assembly 10 and a plug-in assembly 100 configured to selectively engage and disengaged from the control assembly 10. The plug-in assembly 100 can include a patient interface through which medicament can be delivered to the patient. The patient interface can be disposed on the plug-in assembly 100, or, alternatively, can be disposed on a separate infusion set connected to the plug-in assembly 100 via an infusion line.

A medicament container 2 with medicament can be connected to, or be a part of, the drug delivery device 1. The medicament can be moved from the container 2 by the control assembly 10 through the plug-in assembly 100 to the patient. In some aspects, the plug-in assembly 100 can be designed to be disposable, while the control assembly 10 can be designed to be reused with other plug-in assemblies. The plug-in assembly 100 can be connected to the control assembly 10 prior to use and then disconnected from the control assembly 10 after use.

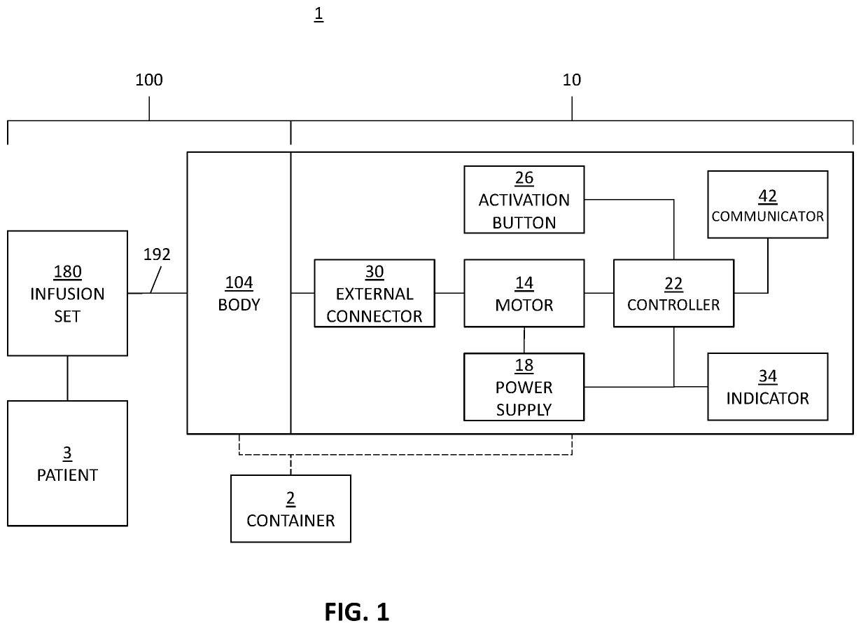

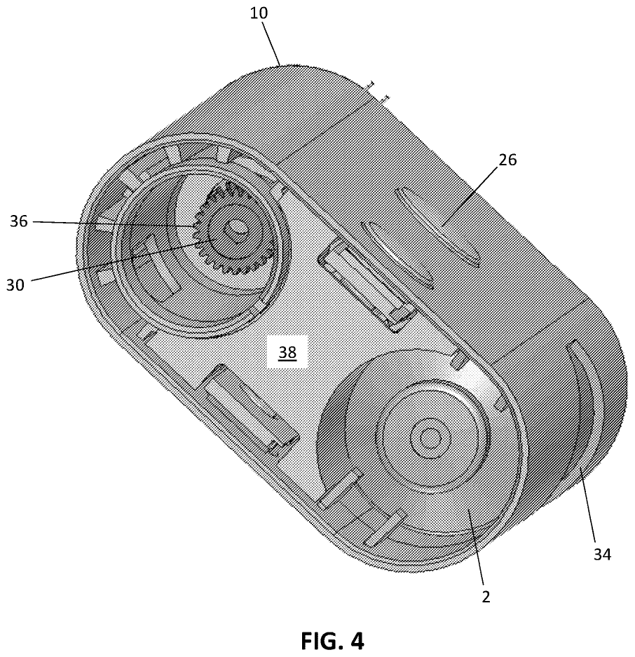

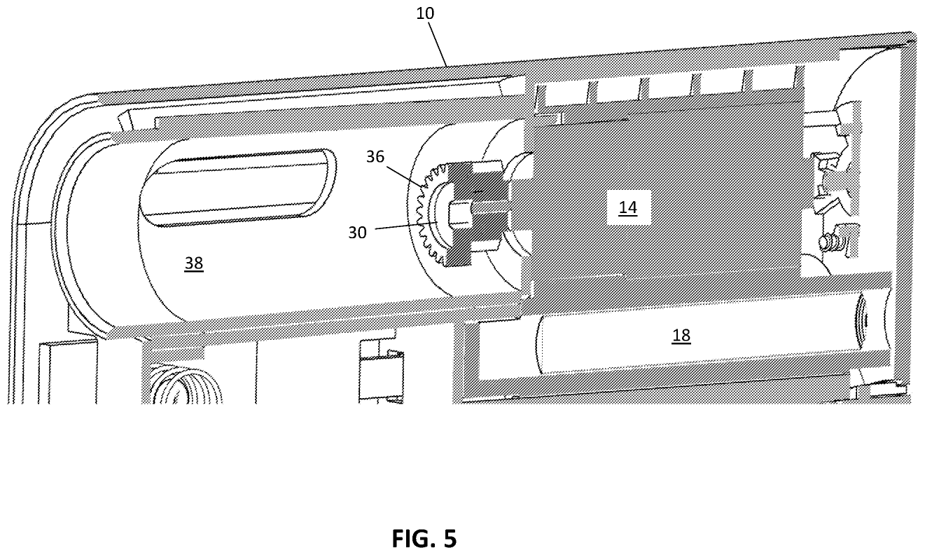

An embodiment of a drug delivery device 1 is depicted in FIGS. 2-10. The drug delivery device 1 includes a control assembly 10 and a plug-in assembly 100. With reference to the embodiment of FIGS. 2-10 and the schematic shown in FIG. 1, the control assembly 10 can include a pump actuator operably connected to a first connector 30. The pump actuator may be a motor 14. The drug delivery device 1 can include any suitable type of motor 14, such as a step motor, a DC motor, a servo motor, or the like. The motor 14 is configured to rotate the first connector 30 in a first rotational direction, a second rotational direction opposite the first rotational direction, or both rotational directions selectively. The motor 14 can receive power from a power supply 18 operably connected thereto. The power supply 18 can include a battery. The battery can be designed to be removable and replaceable or intended to be a non-removable component of the control assembly 10. Operation of the motor 14 can be controlled by a controller 22. The controller 22 can include a processor, a memory, and programmed instructions for operation thereof and of other components in the drug delivery device 1. The control assembly 10 defines a receptacle 38 configured to receive the plug-in assembly 100 therein. The drug delivery device 1 can be operably connected to a patient 3 (shown schematically in FIG. 1) such that one or more medicaments can be transferred from a container 2 to the patient 3 via the drug delivery device 1.

To initiate operation of the drug delivery device 1, a patient 3 (or another individual) may actuate an activation button 26 on the control assembly 10. The activation button 26 may include a push button, a toggle, a switch, a capacitive or resistive touch panel, or the like. The activation button 26 is operably connected to the controller 22, and actuation of the activation button 26 can cause the controller 22 to perform one or more preprogrammed tasks. Tasks can include, but are not limited to, causing the controller 22 to turn on, actuating the motor 14, changing the rotational speed of the motor 14, stopping movement of the motor 14, receiving and/or transmitting data between the drug delivery device 1 and an external computing device, actuating an indicator 34 (described below) to cause an auditory, visual, and/or tactile feedback, or another desired effect.

In some aspects, the drug delivery device 1 can include one or more optional indicators 34 configured to provide feedback to the patient 3. The indicators 34 can be controlled by the controller 22 or be passively operational without need for control. The indicator 34 can be a visual indicator that includes a light-emitting component, such as a light-emitting diode, that can be activated by the controller 22 to turn on a light, turn off a light, sequentially turn on or off a light in a predetermined pattern, and/or change a color of the light.

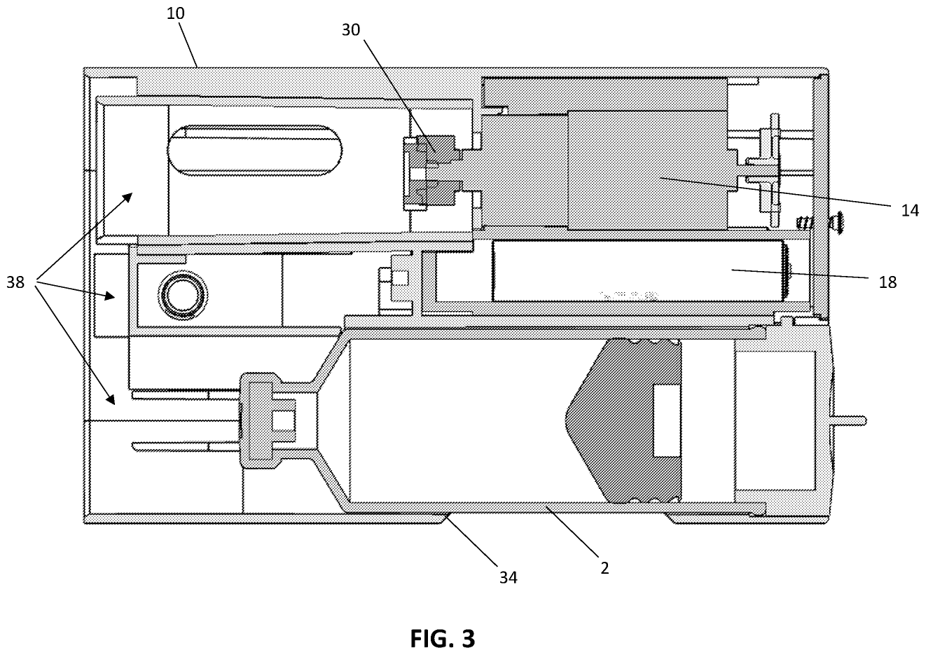

In some aspects, the indicator 34 can include a transparent or semi-transparent window adjacent to the container 2 that allows the patient 3 to view the amount of medicament present in the container 2 (see, e.g., FIG. 3).

In some aspects, the indicator 34 may include an auditory indicator configured to provide a sound indicative of an action or warning to the patient 3. The controller 22 can cause the indicator 34 to provide a sound, stop providing a stop, change the sound provided, or the like.

In some aspects, the indicator 34 can include a tactile indicator configured to provide a tactile response to the patient 3, such as vibration. The controller 22 can cause the indicator 34 to vibrate, stop vibrating, vibrate in a particular pattern, or the like.

In some aspects, the control assembly 10 may optionally include an electronic communicator 42, which may include a transmitter and/or a receiver configured to respectively send and/or receive data to and/or from an external computing device (not shown) pertaining to operation of the drug delivery device 1 and/or the patient 3. The communicator 42 can include components for, and operate according to, known communication protocols, such as Wi-Fi, Bluetooth, NFC, 3G, 4G, 5G, and the like.

The container 2 that contains the medicament can be disposed on or in the drug delivery device 1 or can be separate from the drug delivery device 1 and instead operably connectable to the drug delivery device 1. In some aspects, the container 2 can be disposed in the control assembly 10 (see, e.g., FIG. 3). The container 2 can be designed to not be readily removable from the control assembly 10, or, alternatively, to be removable from the control assembly 10. In embodiments where the control assembly 10 is intended to be reused, the container 2 can be configured for selective engagement and disengagement to and from the control assembly 10. Thus, prior to use, the container 2 (containing the medicament therein) is operably connected to the control assembly 10 such that the drug delivery device 1 can transfer some or all of the medicament from the container 2 to the patient 3. After the transfer or injection is complete, the container 2 can be removed from the control assembly 10. In some aspects, the control assembly 10 can be configured to receive a new container 2 thereafter. The container 2 can include a vial, a syringe, a cannula, a flexible bag or bladder, or the like. In some aspects, the medicament container 2 can be configured to engage with the plug-in assembly prior to being received in the control assembly as will be described later.

The motor 14 operates by rotating a shaft within the motor in a first rotational direction or in a second rotational direction opposite the first rotational direction. The first connector 30 is configured to be engaged with the shaft of the motor 14. When the motor 14 actuates rotation of the shaft in the first rotational direction, the first connector 30 also rotates in the first rotational direction; when the motor 14 actuates rotation of the shaft in the second rotational direction, the first connector 30 also rotates in the second rotational direction. The first connector 30 can include a gear head with a plurality of teeth extending radially from the gear head. In some aspects, the first connector 30 may include one or more protrusions 36 extending therefrom and spaced by gaps 32. The first connector 30 is configured to releasably engage with a second connector 200 as will be described further below.

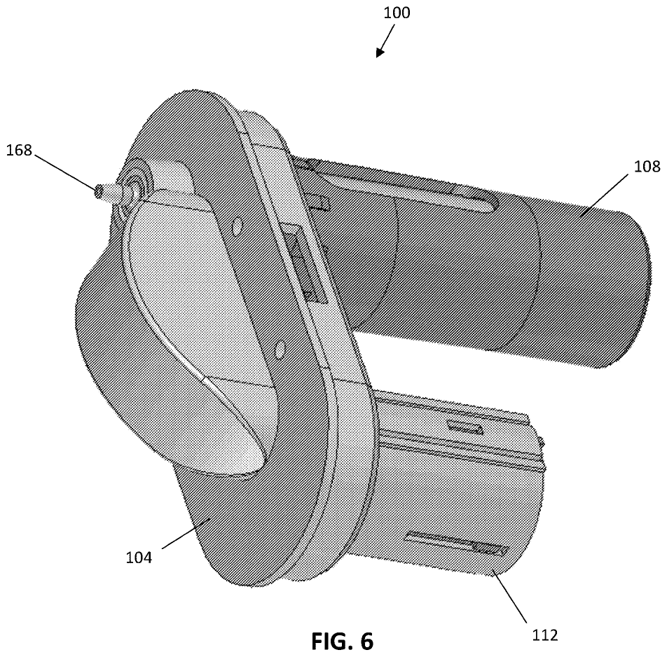

With continued reference to FIG. 1 and with further reference to FIGS. 6-8, the plug-in assembly 100 includes a body 104 and an infusion set 180 connected to the body 104 via an infusion line 192. The infusion set 180 can be any existing infusion set configured to provide an interface with the patient 3 and to deliver a medicament from a source to the patient therethrough. The infusion set 180 includes a contact surface configured to contact the patient 3, for example on the skin of the patient 3. In embodiments where the medicament is to be delivered subcutaneously, intravenously, intramuscularly, or otherwise within the body, the infusion set 180 can include a needle or catheter configured to be inserted into the patient 3. The medicament can be moved through the needle or catheter into the patient 3.

The infusion line 192 can be any commonly used infusion line containing a medical-grade tubing through which the medicament can be moved to the infusion set 180. The infusion line 192 can be connected to the body 104, such that medicament can be moved from the body 104 into and through the infusion line 192 to the infusion set 180.

It should be understood that while FIG. 2 depicts the infusion set 180 separate from the plug-in assembly 100 and connected by the infusion line 192, in some embodiments, the plug-in assembly 100 may include the infusion set 180 (or equivalent components thereto) on the body 104, thus allowing the drug delivery device 1 to be positioned directly at the injection site rather than spaced from a separate infusion set 180 by an infusion line 192.

The body 104 is configured to releasably engage with the control assembly 10. A portion of the body 104 is receivable in the receptacle 38 defined by the control assembly 10. It will be appreciated that the receptacle 38 may be sized and shaped to complement and accommodate the size and shape of the body 104. In some aspects, the body 104 and receptacle 38 may be keyed such that the body 104 can only be received into the receptacle 38 in a desired orientation or direction. This can reduce chances of improper connection of components that would result in leaks, injury to the user, or improper dosing.

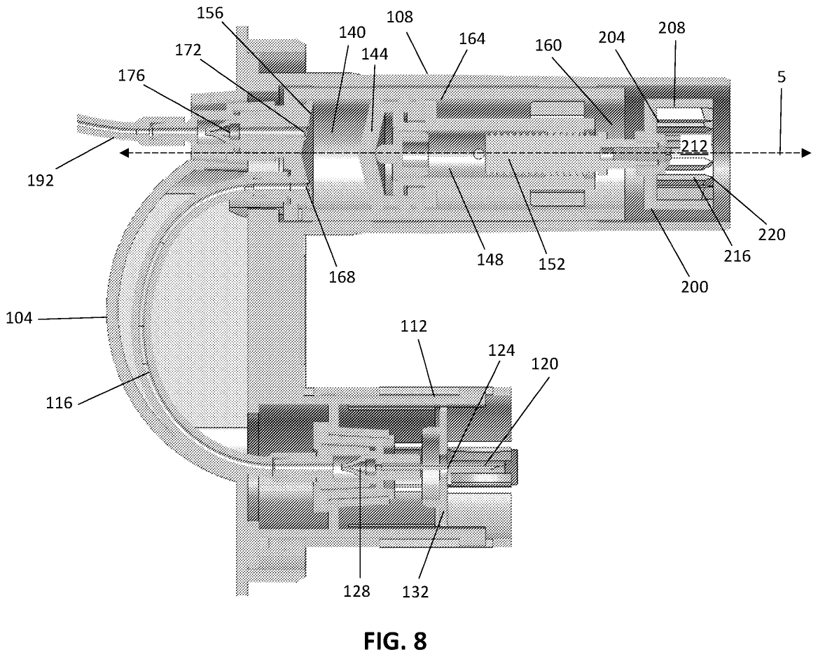

The body 104 includes a pump portion 108 and a needle portion 112 connected to the pump portion 108 via a transfer channel 116. The needle portion 112 includes a transfer needle 120 configured to engage with the container 2. The transfer needle 120 defines a tip at one end thereof configured to be received into the container 2. In some aspects, the tip may be beveled to facilitate piercing of the container 2. As shown in FIG. 8, in some aspects, a needle guard 132 may be disposed on the needle portion 112 to cover the transfer needle 120 to decrease the risk of needle injury to the user. The needle guard 132 may be movable within the needle portion 112 between an open position and a closed position. When the container 2 is not connected to the body 104, the needle guard 132 may be in the closed position, in which the transfer needle 120 is covered. When the container 2 is connected to the body 104, the needle guard 132 may be in the open position, in which the transfer needle 120 is exposed and can be received in the container 2. The needle guard 132 can be movable from the closed position to the open position by the container 2 as the container 2 is being engaged with the body 104. The needle guard 132 may include a resilient member (not shown), such as a spring, configured to bias the needle guard 132 in the closed position, such that when the container 2 is disengaged from the body 104, the resilient member moves the needle guard 132 from the open position back to the closed position to cover the transfer needle 120.

A lumen 124 is defined through the needle 120 and is configured to receive a medicament therein from the medicament container 2. The medicament may be moved through the lumen 124 into the transfer channel 116. A first valve 128 is disposed in the needle portion 108 between the lumen 124 and the transfer channel 116. The first valve 128 may be a one-way flow valve, such as a duckbill valve or another suitable valve type. The first valve 128 is arranged such that a fluid (e.g., the medicament) is permitted to flow in one direction but not in a second opposite direction. As depicted in FIG. 8, the first valve 128 may be arranged such that the medicament may be moved from the lumen 124 into the transfer channel 116 but not from the transfer channel 116 to the lumen 124. Inclusion of the first valve 128 decreases the likelihood of backflow of the medicament during use of the device to prevent medicament or any other substance from being moved back into the transfer needle 120 or the medicament container 2.

The pump portion 108 is connected to the needle portion 112 via the transfer channel 116. The pump portion 108 defines a chamber 140 configured to receive the medicament from the needle portion 112. The chamber 140 is defined by a distal wall 156, a proximal wall 160 spaced opposite the distal wall 156, and a sidewall 164 extending between the distal wall 156 and the proximal wall 160. A chamber inlet 168 on the pump portion 108 fluidly connects the chamber 140 with the transfer channel 116. As shown in the exemplary aspect of FIG. 8, the chamber inlet 168 may be defined on distal wall 156. Fluid (e.g., medicament) can be moved from the needle portion 112, into and through the transfer channel 116, and through the chamber inlet 168 into the chamber 140 of the pump portion 108. The fluid can be moved out of the chamber 140 through a chamber outlet 172. The chamber outlet 172 may be defined on the distal wall 156. The chamber outlet 172 can be in fluid communication with the infusion line 192. A second valve 176 may be disposed between the chamber outlet 172 and the infusion line 192. The second valve 176 may be similar to, or substantially the same as, the first valve 128. The second valve 176 may be a one-way valve, such as a duckbill valve. The second valve 176 is arranged such that fluid can be moved from the chamber 140 through the chamber outlet 172 into the infusion line 192 but cannot be moved from the infusion line 192 into the chamber 140.

Referring to FIG. 8, a piston 148 is movably disposed within the chamber 140 and is configured to be moved axially selectively toward or away from the distal wall 156 of the chamber 140 along a dispensing axis 5. A plunger 144 can be disposed on the piston 148 such that the plunger 144 is between the distal wall 156 and the proximal wall 160. The plunger 144 should be dimensioned such that it is slidable within the chamber 140 while contacting the sidewall 164. It will be understood that the plunger should sufficiently prevent movement of liquid past itself between the plunger 144 and the sidewall 164, such that any liquid in the chamber 140 is kept between the distal wall 156 and the plunger 144. The plunger 144 may include any suitable materials, coatings, films, laminations, and/or treatments deemed useful or necessary with respect to the particular fluid or medicament that will contact the plunger 144. The piston 148 can be moved via various mechanism, for example, as depicted in FIG. 8, via a screw 152 having threads thereon. The screw 152 is rotatable around the dispensing axis 5 and can be axially fixed along the dispensing axis 5. The piston 148 may include complementary threads configured to engage with those of the screw 152, such that when the screw 152 is rotated in a first direction about the dispensing axis 5, the piston 148 is moved toward the distal wall 156, and when the screw 152 is rotated in a second direction opposite the first direction, the piston 148 is moved toward the proximal wall 160. The screw 152 can be actuated by the motor 14 (described above and depicted in FIGS. 2 and 3).

The screw 152 can include, or be operably connected to, a second connector 200. The second connector 200 is configured to engage with the first connector 30 described above when the plug-in assembly 100 is connected to the control assembly 10. When engaged, the second connector 200 is rotatable by the motor 14 via the first connector 30. The second connector 200 is connected to the screw 152 such that the proximal wall 160 is between the screw 152 and the connector 200. The screw 152 may be entirely within the chamber 140, while the connector 200 is outside of the chamber 140. Such an arrangement allows for the chamber 140 to maintain a clear or sterilized environment relative to the external environment.

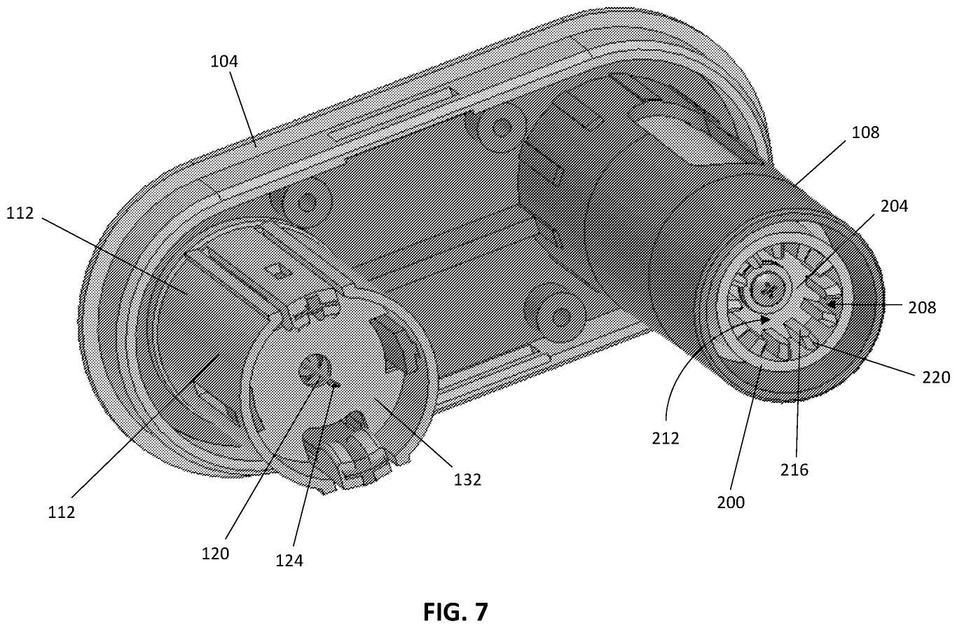

Referring to the exemplary aspect of FIGS. 7 and 8, the second connector 200 may include one or more protrusions 216 thereon separated by gaps 208. The protrusions 216 are configured to engage with one or more protrusions 36 on the first connector 30. When the first and second connectors 30 and 200 engage with one another, the protrusions 36 are received into the gaps 208, and the protrusions 216 are received into the gaps 32 to rotationally fix the first and second connectors 30 and 200 relative to one another. As such, when the two connectors 30 and 200 are engaged, and the first connector 30 is rotated by the motor 14, the second connector 200 is also rotated in the same rotational direction. In some aspects, each protrusion 216 may terminate in a tapered end 220 configured to facilitate alignment with the respective protrusions 36 on the first connector 30.

One or more fluids may be introduced into the chamber 140 from the medicament container 2 during use. The one or more fluids may include one or more medicaments. When the container 2 is engaged with the needle portion 112 and the transfer needle 120 is connected to the container 2, a fluid pathway is established between the container 2 and the chamber 140 via the transfer channel 116 as described above.

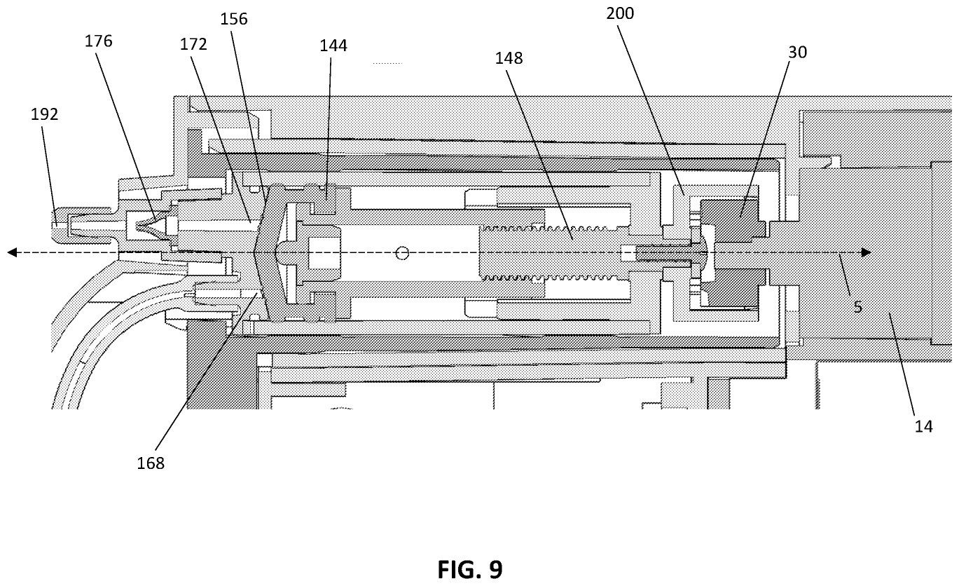

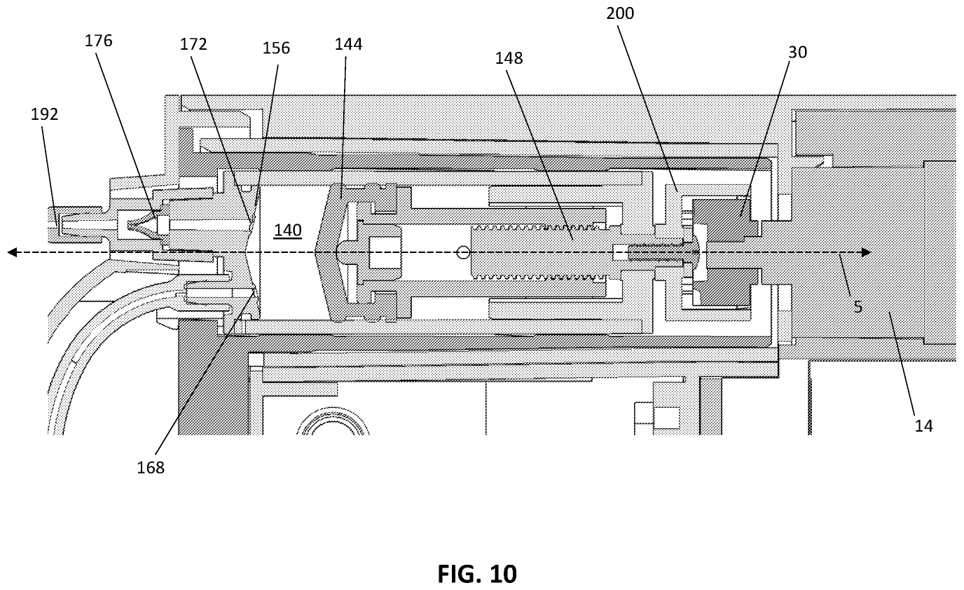

Referring to FIG. 9, when the plug-in assembly 100 is connected to the control assembly 10, the piston 148 may first be positioned within the chamber 140 such that the plunger 144 contacts the distal wall 156 and substantially no space is defined between the plunger 144 and the distal wall 156. Movement of the piston 148 in a first axial direction along the dispensing axis 5 away from the distal wall 156 creates a vacuum within the chamber 140 between the distal wall 156 and the plunger 144 as shown in FIG. 10. The vacuum causes fluid from the container 2 to be moved out of the container 2, through the lumen 124 of the transfer needle 120, past the first valve 128, through the transfer channel 116, and into the chamber 140 through the chamber inlet 168. It will be understood that the further the piston 148 is moved away from the distal wall 156, the more vacuum is created in the chamber 140, and thus the more fluid is moved therein. It will be noted that although the chamber 140 also include a chamber outlet 172, fluid is not introduced into the chamber 140 through the chamber outlet 172 during this process due to the presence of the second valve 176, which prevents fluid from moving in the direction toward the chamber 140.

After the desired amount of fluid has been moved into the chamber 140, the piston 148 can be moved in a second axial direction opposite the first axial direction along the dispensing axis 5. Movement of the piston 148 causes the fluid in the chamber 140 to be compressed between the distal wall 156 and the plunger 144 and causes the fluid to be moved towards and into the chamber outlet 172. The fluid is moved out of the chamber 140 through the chamber outlet 172 and past the second valve 176 into the infusion line 192. It will be appreciated that some of the fluid may be forced back into the transfer channel 116 via the chamber inlet 168, but the fluid is precluded from moving back into the lumen 124 or into the medicament container 2 past the first valve 128, which prevents movement of fluid in this direction.

The distance that the piston 148 is moved in the first and/or second axial directions can depend on the desired amount of fluid to be administered into the infusion line 192 to the patient. It will further be understood that the rate and timing of the movement of the piston 148 can be controlled by the controller 22 described above and can be adjusted based on the desired administration parameters. The chamber 140 may have a smaller volume than that of the container 2 or that of the total volume of the desired administration of fluid to the patient. In such scenarios, movement of the piston 148 in the first and then second axial directions can be repeated until the desired amount of fluid has been administered.

The drug delivery device 1 may be designed such that the container 2 is preloaded therein and the user need not insert the container 2 prior to use. Alternatively, the container may be provided separate from the control assembly 10 and the plug-in assembly 100. In such scenarios, the user must connect the container 2 to the drug delivery device 1 prior to engaging the plug-in assembly 100 with the control assembly 10. In some aspects, such as the exemplary embodiments depicted in FIGS. 3-10, the container 2 can be connected to the control assembly 10. After the container 2 is engaged with the control assembly 10, the plug-in assembly 100 can be engaged with the control assembly 10, and the transfer needle 120 can fluidly connect with the container 2 as described previously.

The medicament can be moved from the plug-in assembly 100 through the depicted infusion line 192 to the connected infusion set 180, which can be placed at the desired injection site. The infusion set 180 can include a contact surface configured to contact the patient and a needle or catheter configured to penetrate the patient's body and through which the medicament enters the patient. In some aspects, such as shown in FIG. 2, the infusion set 180 can be separated from the body 104 by an infusion line 192, which can be a flexible tube or catheter. In such aspects, the infusion set 180 is flexibly connected to the plug-in assembly 100, which allows for easier and/or more convenient placement of the infusion set 180 onto the patient's body relative to the plug-in assembly 100. It will be understood that the longer and more flexible the infusion line 192 is, the more flexibility there is in where the infusion set 180 is positioned on the patient relative to the body 104. Although FIG. 2 depicts the drug delivery device 1 having an infusion set 180 separate from the plug-in assembly 100, it should be understood that in some aspects, the drug delivery device 1 can be designed to have a patient contact surface and needle or catheter on the plug-in assembly 100 itself rather than connecting to a separate infusion set 180 via an infusion line 192.

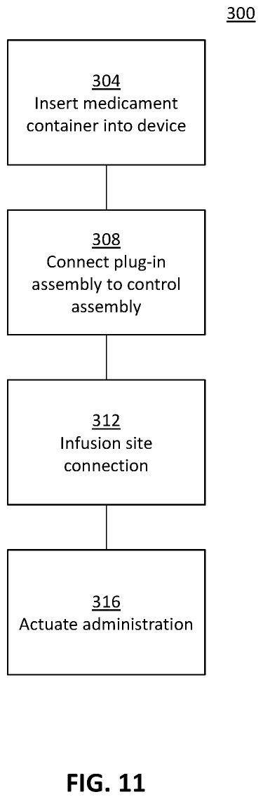

Referring to FIG. 11, an exemplary infusion process 300 is depicted. While the process 300 is described with reference to components of the exemplary drug delivery device 1, the steps of the process 300 can refer to components of any of the embodiments described herein. In step 304, a user introduces a container 2 into the drug delivery device 1. The container 2 can contain one or more medicaments for infusion. In some aspects, the container 2 can be introduced into the control assembly 10; in other aspects as will be described below, the container 2 can be introduced into the plug-in assembly.

In step 308, the plug-in assembly 100 is engaged with the control assembly 10 such that the first connector 30 is engaged with the second connector 200. At this stage, the motor 14 is operably connected to the piston 148, and the container 2 is in fluid communication with the transfer needle 120. The drug delivery device 1 is thus ready to deliver the medicament from the container 2.

In step 312, the infusion line 192 and connected infusion 180 set can be connected to the plug-in assembly 100. It will be understood that the drug delivery device 1 can be used with different types of infusion lines and infusion sets, and this disclosure does not limit the applicability of different connectable infusion mechanisms. The infusion set 180 can be positioned on the desired infusion spot on the body. It will be appreciated that the step of securing the infusion set 180 to the infusion site can be completed prior to step 312. In some aspects, the drug delivery device can include an infusion set, or equivalent features thereof, on the plug-in assembly. In such aspects, the entire drug delivery device can be placed on the desired infusion site, and a separate infusion line and infusion set may not be required.

In step 316, the user can actuate infusion by actuating the activation button 26 described above. When the activation button 26 is actuated, the controller 22 activates the motor 14 to rotate the first connector 30. As the first connector 30 rotates, the operably connected second connector 200 also rotates, and the piston 148 is moved axially in the first axial direction within the plug-in assembly 100, causing medicament from the medicament container 2 to be moved into the chamber 140. The controller 22 may include preprogrammed instructions that dictate how far the piston 148 should be moved in the first axial direction (and thus how much medicament is extracted from the container 2). Once the intended amount of medicament has been extracted, the controller 22 causes the motor 14 to rotate in the opposite rotational direction, thus causing the piston 148 to move in the second axial direction to expel the medicament from the chamber 140 out through the chamber outlet 172. The steps of moving the piston 148 in the first direction to extract medicament from the container 2 and then moving the piston 148 in the second direction to expel the medicament can be repeated an intended number of times, and the controller 22 can include programmed instructions for doing so. When the infusion process is completed, the controller 22 can transmit a signal to the indicator 34 to provide a notification to the user that infusion is complete.

In some aspects, step 316 can be separated into various sub-steps controllable by the user. For example, the user may actuate the button 26 to move the piston 148 in the first axial direction, actuate the same (or a different) button to then cause the piston 148 to move in the second axial direction, and/or actuate the same (or different) button to cause the piston 148 to again move in the first axial direction. This could allow the user to control the amount of medicament being delivered and/or the rate at which the medicament is being delivered.

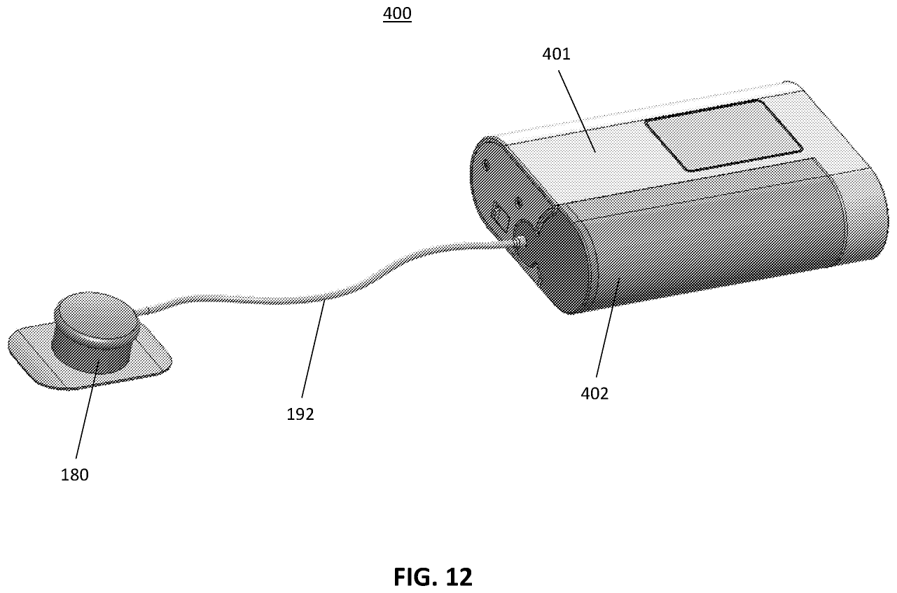

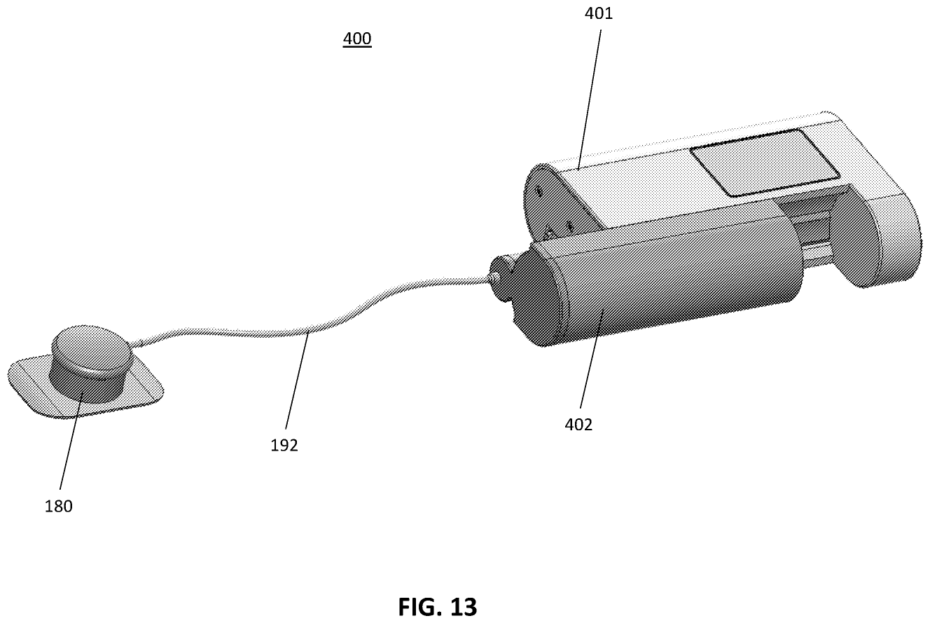

In some aspects, it may be advantageous to engage the medicament container 2 with the plug-in assembly 100 prior to coupling the plug-in assembly 100 with the control assembly 10. Referring to the exemplary aspect of FIGS. 12-14, a drug delivery device 400 is shown having a plug-in assembly 402 that is configured to receive a container 2 therein. The plug-in assembly 402 is configured to engage with a control assembly 401. Various components of the drug delivery device 400 may be similar to, or substantially the same as, components described above with respect to the drug delivery device 1. The control assembly 401 may include at least the same features of the control assembly 10.

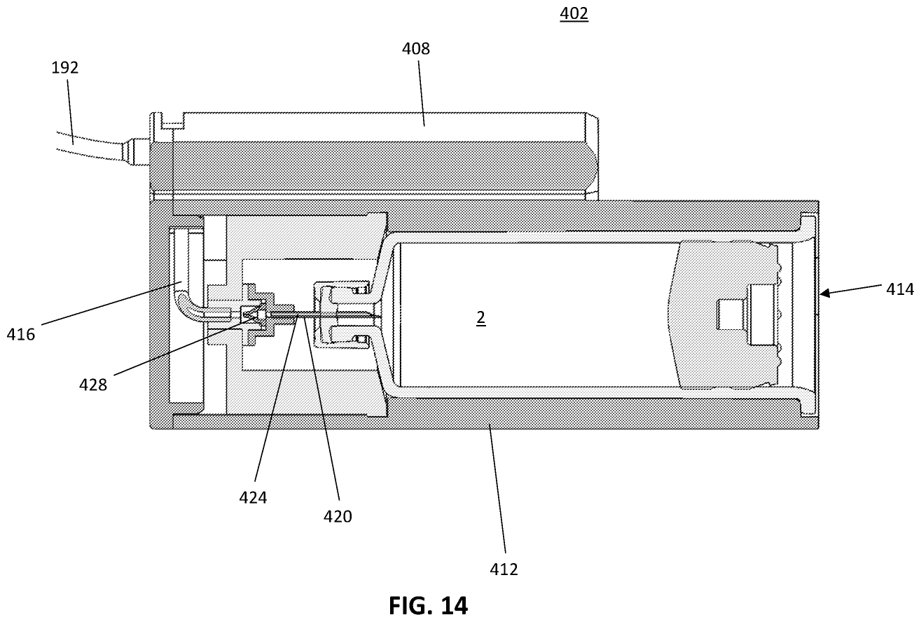

Referring to FIG. 14, the plug-in assembly 402 of the drug delivery device 400 includes a pump portion 408 and a needle portion 412. The pump portion 408 can be similar to, or substantially the same as, the pump portion 108 described above. The needle portion 412 can define a receptacle 414 configured to receive therein the container 2. When the container 2 is inserted into the receptacle 414, a transfer needle 420 pierces the container 2 and forms a fluid path between the interior of the container 2 and a lumen 424 extending through the transfer needle 420. Fluid from the container 2 can be moved through the lumen 424, past a first valve 428, through a transfer channel 416, and into the pump portion 408 in a manner similar to what is described above with respect to the drug delivery device 1.

The mechanics of moving fluid from the container 2 to the infusion line 192 via the pump portion 408 is substantially the same as described in previous embodiments. Receiving the container 2 within the plug-in assembly 402 may simplify use and decrease reusable surfaces that need to be sterilized, cleaned, and/or maintained. In use, a user can insert the container 2 into the plug-in assembly 402 first, and then engage the plug-in assembly 402 with the control assembly 401. After infusion is completed, the plug-in assembly 402 can be disengaged from the control assembly 401 and disposed of along with the container 2 therein, while the control assembly 401 can be cleaned and reused in another infusion.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

1. A drug delivery device for administering a medicament to a patient, the drug delivery device comprising:
a control assembly having a first connector and a motor configured to rotate the first connector; and
a plug-in assembly having a transfer needle configured to receive the medicament therein and a pump portion having a chamber configured to receive the medicament from the transfer needle, a piston movable within the chamber, and a second connector operably connected to the piston,
wherein the plug-in assembly is removably couplable to the control assembly, such that when the plug-in assembly is coupled to the control assembly, the first connector is operably coupled to the second connector, and
wherein when the plug-in assembly is coupled with the control assembly, and when the motor rotates the first connector, the second connector also rotates, and the piston is moved axially within the chamber.

2. The drug delivery device of claim 1, further comprising a patient interface disposed at an injection site, the medicament being movable through the patient interface to the patient.

3. The drug delivery device of claim 2, wherein the patient interface includes an infusion set having a needle insertable into the injection site, the infusion set being connected to the plug-in assembly via an infusion line.

4. The drug delivery device of claim 1, wherein the motor is configured to rotate the first connector in a first rotational direction to cause the piston to be moved in a first axial direction and in a second rotational direction opposite the first rotational direction to cause the piston to be moved in a second axial direction opposite the first axial direction, and
wherein movement of the piston in the first axial direction causes the medicament to be moved into the chamber, and movement of the piston in the second axial direction causes the medicament to be expelled from the chamber.

5. The drug delivery device of claim 1, wherein the piston is entirely within the chamber.

6. The drug delivery device of claim 1, wherein the plug-in assembly includes a first valve between the transfer needle and the chamber, the first valve being configured to allow the medicament to be moved from the transfer needle to the chamber but not from the chamber to the transfer needle.

7. The drug delivery device of claim 1, wherein the chamber is configured to receive the medicament through a chamber inlet and to expel the medicament through a chamber outlet, the plug-in assembly having a second valve configured to allow the medicament to be moved out of the chamber through the chamber outlet but not into the chamber through the chamber outlet.

8. A drug delivery system comprising:
a control assembly having a first connector and a motor configured to rotate the first connector;
a plug-in assembly having a transfer needle configured to receive a medicament therein, a chamber configured to receive the medicament from the transfer needle, a piston movable within the chamber, and a second connector operably connected to the piston; and
a medicament container having the medicament therein and being couplable with the plug-in assembly such that the transfer needle is in fluid communication with the medicament in the medicament container; and
an infusion patient interface configured to contact an injection site and to deliver the medicament to the injection site from the chamber,
wherein the plug-in assembly is removably couplable to the control assembly, such that when the plug-in assembly is coupled to the control assembly, the first connector is operably coupled to the second connector, and
wherein when the plug-in assembly is coupled with the control assembly, and when the motor rotates the first connector, the second connector also rotates, and the piston is moved axially within the chamber.

9. The drug delivery system of claim 8, wherein the medicament container has a first volume, the chamber has a second volume, and the first volume is greater than the second volume.

10. The drug delivery system of claim 8, wherein the medicament container is configured to be engaged with the plug-in assembly prior to the plug-in assembly being coupled with the control assembly.

11. The drug delivery system of claim 8, wherein the medicament container is configured to be engaged with the control assembly prior to the plug-in assembly being coupled with the control assembly.

12. The drug delivery system of claim 8, further comprising a first valve between the medicament container and the chamber and a second valve between the chamber and the infusion patient interface,
wherein the first valve is configured to allow the medicament to be moved from the medicament container to the chamber but not from the chamber to the transfer needle, and the second valve is configured to allow the medicament to be moved from the chamber to the infusion patient interface but not from the infusion patient interface into the chamber.

13. The drug delivery system of claim 8, wherein the plug-in assembly and the control assembly are shaped such that the plug-in assembly can be engaged with the control assembly in only one orientation.

14. The drug delivery system of claim 8, configured to transmit a signal to a computing device indicative of operation of the drug delivery system.

15. A method of injecting a medicament using a drug delivery device, the drug delivery device having a plug-in assembly removably couplable to a control assembly, the method comprising:
introducing a medicament container into the drug delivery device;
coupling the plug-in assembly to the control assembly such that a first connector on the control assembly is operably engaged with a second connector on the plug-in assembly, and such that a transfer needle on the plug-in assembly is in fluid communication with the medicament in the medicament container; and
actuating a motor to cause rotation of the first connector in a first rotational direction.

16. The method of claim 15, further comprising actuating the motor to cause rotation of the first connector in a second rotational direction opposite the first rotational direction.

17. The method of claim 15, further comprising disposing a patient interface onto an injection site and coupling the patient interface with the plug-in assembly,
wherein the patient interface being configured to receive the medicament from the plug-in assembly and to transfer the medicament to the injection site.

18. The method of claim 15, wherein introducing the medicament container includes coupling the medicament container with the control assembly prior to coupling the control assembly with the plug-in assembly.

19. The method of claim 15, wherein introducing the medicament container includes coupling the medicament container with the plug-in assembly prior to coupling the plug-in assembly with the control assembly.

20. (canceled)
